(19) United States
(12) Patent Application Publication
(10) Pub. No.: US 2004/0094163 A1
(43) Pub. Date: May 20, 2004

(54) Male sexual aid device

(76) Inventor: Bradley Benson, Gibsons (CA)

Correspondence Address: Paul Smith Intellectual Property Law, 330-1508 West Broadway, Vancouver, BC, V6JIW8, CA

(21) Appl. No.: 10/471,461

(22) PCT Filed: Apr. 24, 2002
(86) PCT No.: PCT/CA02/00606

(30) Foreign Application Priority Data
May 2, 2001 (CA) 2346188

(51) Int. Cl. A61F 6/04; A61F 6/02

(52) U.S. Cl. 128/844; 128/842

(57) ABSTRACT

A male sexual aid device having the general shape of a condom, but which is loose fitting and which has a tail for insertion between the buttocks of the user in order to retain the device in place. The device can be used in masturbation in order to stimulate vaginal penetration or for sexual intercourse. The device provides added sexual stimulation to the user's penis and anal area and may be especially useful for those suffering from various forms of sexual dysfunction. In some embodiments, the device provides a form of birth control and protection from the transmission of STDs.

### FIELD OF THE INVENTION

This invention relates to a male sexual aid that can be used for the purposes of masturbation, primarily for those men with ejaculatory and/or erection difficulties, or as a condom during sexual intercourse.

### BACKGROUND OF THE INVENTION

Condoms are used throughout the world for the prevention of sexually transmitted diseases (STDS) and/or for the prevention of conception. However, the typical condom as generally known to the overall population contains several shortcomings. Specifically, condoms mainly cover the male genitalia only to the approximate base of the penis, allowing the possible transmission of some STDs such as Human Papilloma Virus (HPV) or Herpes Simplex Virus (HSV) to shed through the uncovered skin at the base of the penis and/or scrotum. In addition, typical condoms can leak at the base or can slip off during or following intercourse without the knowledge of the partners, which can lead to the transmission of STDs and/or conception. In addition, during the application or removal of the typical condom, as it is rolled or unrolled along the penile shaft, the user's pubic hair may become painfully entangled. In addition, condom use is known to lessen sexual sensations due to the glove effect of the condom itself.

One of the most common forms of male sexual dysfunction is erectile dysfunction, sometimes known as impotence, and is defined as the inability to maintain an erection sufficient for intercourse. Male sexual dysfunction may also arise from an impaired ability to feel or interpret sexual sensations in the penis caused by nerve damage or psychological trauma.

Erectile dysfunction can occur for many reasons; however, most often it is a combination of psychological and physical reasons. The most frequent physical causes of erectile dysfunction are from diseases that affect the blood vessels, the most common of which are arteriosclerosis and diabetes. Diseases that affect the nervous system, such as diabetes, multiple sclerosis and alcoholism, can also impact the ability to have an erection. Spinal cord injuries that affect nerve impulse transmission due to the destruction of nerve fibers are another cause of erectile dysfunction. Certain surgeries, radiation therapy and hormonal abnormalities can contribute to the problem as well. On the psychological side, stress and anxiety due to job, relationship or financial difficulties are also commonly found in men with erectile dysfunction. Psychological influences have a cumulative effect. Any physical or emotional problem that affects the man's veins, arteries, nerves, penile or pelvic anatomy or hormone status can alter the ability to have an erection or orgasm. As well, as men age it is normal for many men to experience sexual dysfunctions.

Studies show that erectile dysfunction effects over 50% of men between the ages 40 and 70 in the United States. At age 70, the figure climbs to 67%, affecting more than 22 to 30 million men according to studies by the United States National Institutes of Health.

In addition to the problems these conditions present for sexual intercourse between two persons, these problems may also impair the ability to enjoy sexual sensations during self or partner assisted stimulation, and may impair the ability to achieve orgasm. Furthermore, masturbation and orgasm are very important in the personal lives of men. American researcher Alfred Kinsey and others have estimated that at mid 20^{th} century at least 92% of all American males and 70 to 80% of all females have indulged in masturbation, and European studies show comparable figures.

Masturbation is defined as the manipulation of the genital organs for pleasure, usually to orgasm. The term masturbation generally connotes self manipulation, but it can also be used to describe manipulation of or by a sexual partner, exclusive of sexual intercourse. Once the object of extravagant superstitions and severe taboos, masturbation by adults was frowned upon in the majority of pre-modern societies and is still considered immoral by many.

The stigma against masturbation is decreasing, and many students of sexual behavior extol its virtues as being healthy, pleasurable, sedative, and a release of tension. Masturbation is a widely used element in sex therapy and is being promoted as beneficial in the context of ‘safe sex’ in today's society.

Most masturbation aids available to the public are re-usable latex replicas of female vaginas and can be embarrassing for men to own, need to be cleaned after each use, and are potentially unsanitary. The device of the present invention is not an obvious sexual aid, which eliminates the embarrassment of ownership, and because it is disposable, eliminates the risk of infection and the need for cleaning.

For some men, falling to sleep is a problem, particularly as they age. In many cases, this problem can be overcome through the attainment of a sexual orgasm, which generates a sense of well being and relaxation, conditions that in turn can often lead immediately to sleep. It is therefore an object of this invention to provide a male sexual aid, which can be comfortably worn and will stay in place all night long, to allow a man to drift to sleep after orgasm without the necessity of attending to the ejaculate and interrupting this transition.

It is a further object of this invention to obviate the need to roll or unroll the device along the penis during application thereby avoiding potentially painful entanglement in the user's pubic hair.

Another object of the invention is to provide a source of added sexual stimulation for those users suffering from sexual dysfunction; specifically, erectile dysfunction, delayed ejaculation, or impaired feelings of sexual sensations.

It is a further object of an embodiment of the present invention to provide a sufficient barrier to prevent the transmission of STDs and to prevent conception.

### SUMMARY OF THE INVENTION

This invention provides a male sexual aid device. The preferred embodiment of the device comprises a sheath of flexible material for covering the penis of a user, the sheath having a closed front end and a rear end with an opening therein. A mid-portion extends from the rear end of the sheath for covering the scrotum of a user and an elongated tail extends from the mid-portion for insertion between the buttocks thereby retaining the device in position on the user.

In another embodiment, the device comprises a sheath of flexible material for covering the penis of a user, the sheath having a closed front end, a mid-portion and an elongated tail. Two openings are located in the mid-portion, one on the top and one on the bottom. In use, the user's penis is inserted through the top opening into the sheath and the user's scrotum is placed through the top and bottom openings. The elongated tail extends from the mid-portion for insertion between the buttocks of the user.

In one aspect of the invention, the sheath, mid-portion and tail are integrally formed of a flexible material.

In another aspect, the sheath and mid-portion are integrally formed of a flexible material and the tail is formed of a different material, which is attached to, or blended with, the mid-portion.

In another aspect, the mid-portion is in the shape of a pouch for the scrotum.

The flexible material may be a plastic, or a latex rubber, polyurethane or other viral blocking material.

In yet another aspect of the invention, the tail is provided with a plurality of spaced apertures.

A method according to the invention comprises installing and retaining the preferred embodiment of the invention by inserting the user's penis into the sheath and inserting the elongated tail between the user's buttocks.

In one aspect of the invention, the device is installed and retained by connecting a stimulatory anal type plug to the device by releasably inserting said plug in the appropriate spaced aperture located in the tail of the device. The user's penis is then inserted into the sheath and the anal plug is inserted into the user's anus.

In another of its aspects, the invention comprises a sexual aid kit comprising a male sexual aid device with a sheath, a mid-portion and an elongated tail, said tail being provided with a plurality of spaced apertures, and a stimulatory anal type plug for attachment to said device and insertion in the anus of a user of said device, said plug having a portion dimensioned to be releasably inserted into said aperture.

Further aspects of the invention will be appreciated by reference to the description of embodiments of the invention below and to the claims.

### BRIEF DESCRIPTION OF THE DRAWINGS

The preferred embodiment of the invention will be described by reference to the accompanying drawings, in which:

FIG. 1 is a side view of the preferred embodiment of the invention.

FIG. 2 is a side view of a second embodiment of the invention.

FIG. 3 is a side view of a third embodiment of the invention.

FIG. 4 is a top view of a fourth embodiment of the invention.

FIG. 5 is a side view of a variation of the embodiment of the invention shown in FIG. 1.

FIG. 6 is a side view of a variation on the embodiment shown in FIG. 3.

FIG. 7 is a top view of a variation on the embodiment shown in FIG. 4.

FIG. 8 is a profile view of the trunk of a body of a user with one leg cut away to illustrate the positioning on the body of the preferred embodiment shown in FIG. 1.

FIG. 9 is a profile view of the trunk of a body of a user with one leg cut away to illustrate the positioning on the body of the preferred embodiment shown in FIG. 4.

FIG. 10 is a side view of a stimulatory type plug for use with embodiments of the invention.

### DETAILED DESCRIPTION OF THE PREFERRED AND ALTERNATIVE EMBODIMENTS OF THE INVENTION

In FIG. 1, reference numeral 10 generally indicates a male sexual aid device comprising a sheath 12, a mid-portion 14 and an elongated tail 16. The device 10 may be of any suitable flexible material such as plastic, latex or polyurethane.

The sheath 12 has a closed front end 18 and a rear end 20 having an opening 22 therein. The sheath 12 accommodates the penis of the user and may be provided in different sizes to accommodate different users.

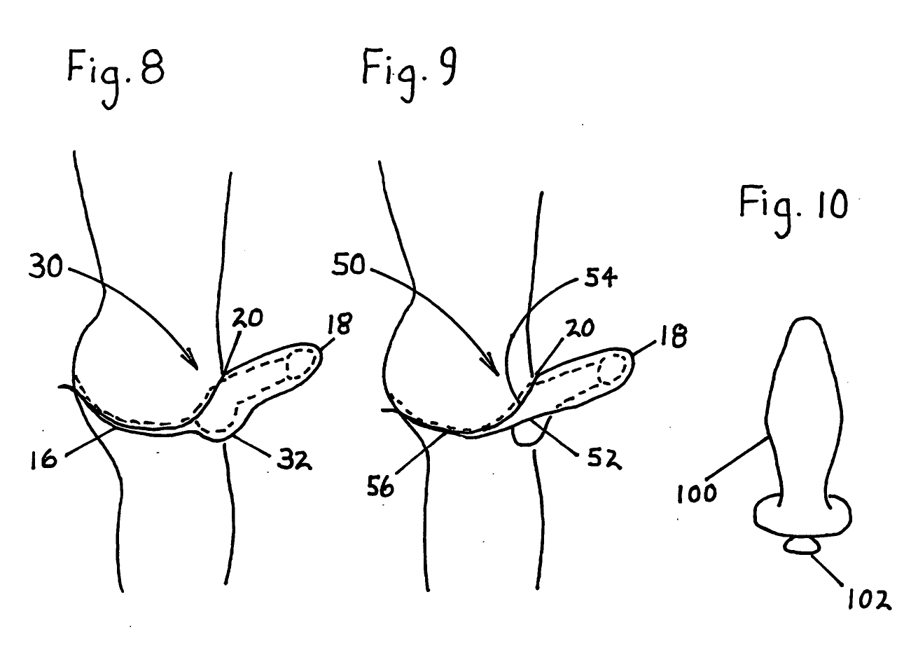

Mid-portion 14 extends from the rear end 20 of the sheath 12 for covering the scrotum of a user. In the preferred embodiment of FIG. 1, the tail 16 extends from and is a continuation of the mid-portion 14. In use, the tail 16 extends over the anus and between the buttocks, as shown in FIGS. 8 and 9. It is through the placement of the tail 16 between the buttocks that the device 10 is retained in position on the user.

When the sheath 12 is in place over the penis, the closed end 18 covers the glans penis and the rear end 20 comes to rest at the base of the penis, in which position it is retained through the action of the tail 16.

The sheath 12 is of a diameter and length such that it loosely covers the entire penis. The loose nature of the sheath 12 allows for ease of placement over the male genitalia and provides for the smooth movement of the penis within the sheath 12, which simulates the vaginal environment and allows an increase in sexual stimulation for the user whether being used as a condom device or as a masturbation device.

The device 10, when used as a condom, is preferably of latex rubber or polyurethane, for those with latex allergies, or an alternate material that acts as a mechanical barrier against the diffusion of agents which may cause STDs and also acts as a barrier to semen to prevent conception.

The mid-portion 14 covering the scrotum provides a mechanical barrier between the skin of a user and a partner so that sexually transmitted diseases cannot be transferred by means of viral shedding through the skin or from open lesions and bodily fluids. The mid-portion 14 may also provide added sexual stimulation to the scrotum.

The tail 16 extends over the anus between the buttocks, where it is retained in place through the combination of the tension of the buttocks and the friction between the material and the skin, which friction may be increased by the adhesive effect of perspiration. When tail 16 is firmly secured in place, it acts to retain the rear end 20 in its position at the base of the penis. In this manner, the tail 16 acts as a retention device to counteract slippage of the sheath 12 during and after use. The tail 16 may also provide added sexual stimulation to the user's anal area.

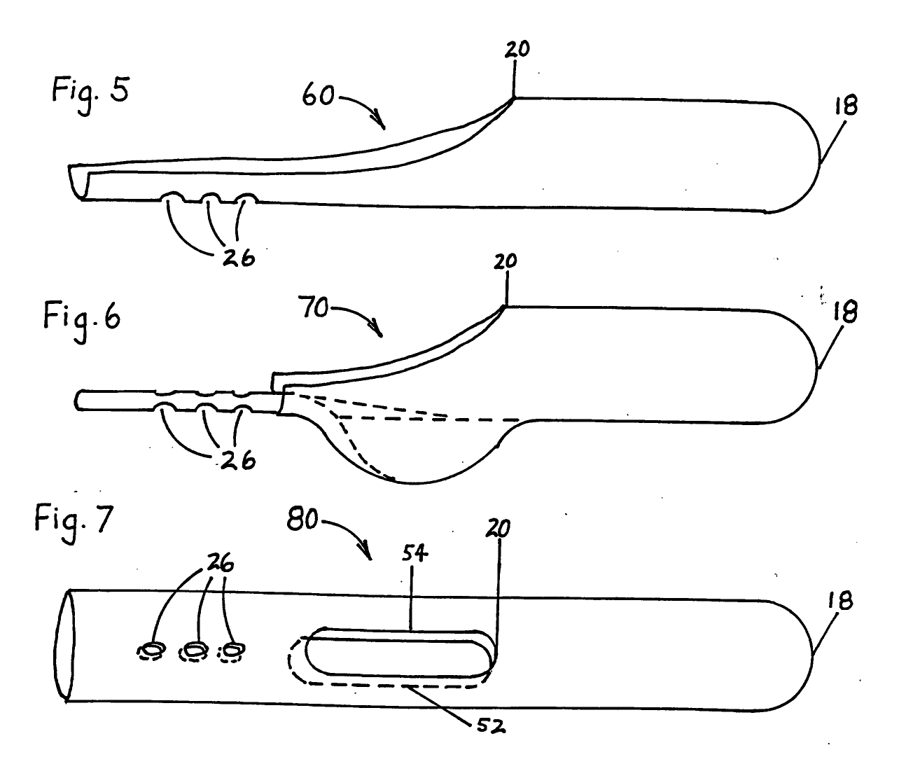

As an alternative to being secured between the buttocks by the combination of tension and friction, and to impart additional sexual sensation to the user, the tail may be adapted to be secured in the opening of the anus. FIGS. 5, 6 and 7 show various embodiments of the invention in which a plurality of spaced apertures 26 are present. A stimulatory anal type plug 100, as shown in FIG. 10, may be releasably connected to the device (60, 70 or 80) and inserted into the anus thereby retaining the device (60, 70 or 80) in place on the body of a user with the rear end 20 in position at the base of the penis. The stimulatory anal type plug 100 is releasably connected to the device by way of a button 102, which is placed in the one of the apertures 26 best positioned to accommodate the size of the user.

As stated above, the sheath is designed to fit loosely over the penis. The typical commercially available condom tends to reduce the overall stimulation of the penis, whereas the loosely fitting sheath, when used with or without internal lubrication, may enhance sexual stimulation and simulate vaginal penetration as movement of the penis within the sheath occurs.

The device may be used in masturbation with or without internal lubrication to enhance sexual stimulation and may be of variable loose fit to simulate the sensation of vaginal penetration. The device may also be used in some cases of erectile dysfunction to stimulate the penis to a sufficient degree of erection to achieve vaginal penetration, provided the user is physically capable of achieving an erection in the first place. In this manner, the device may benefit men who suffer from sexual dysfunction and are unable to achieve or maintain an erection adequate for sexual intercourse, or benefit men whose ability to sense sexual stimulation in the penis has been impaired through nerve damage.

In the preferred embodiment of FIG. 1, the mid-portion forms a generally linear extension of the lower side of the sheath, with the upper side being cut away in tapering fashion towards the tail.

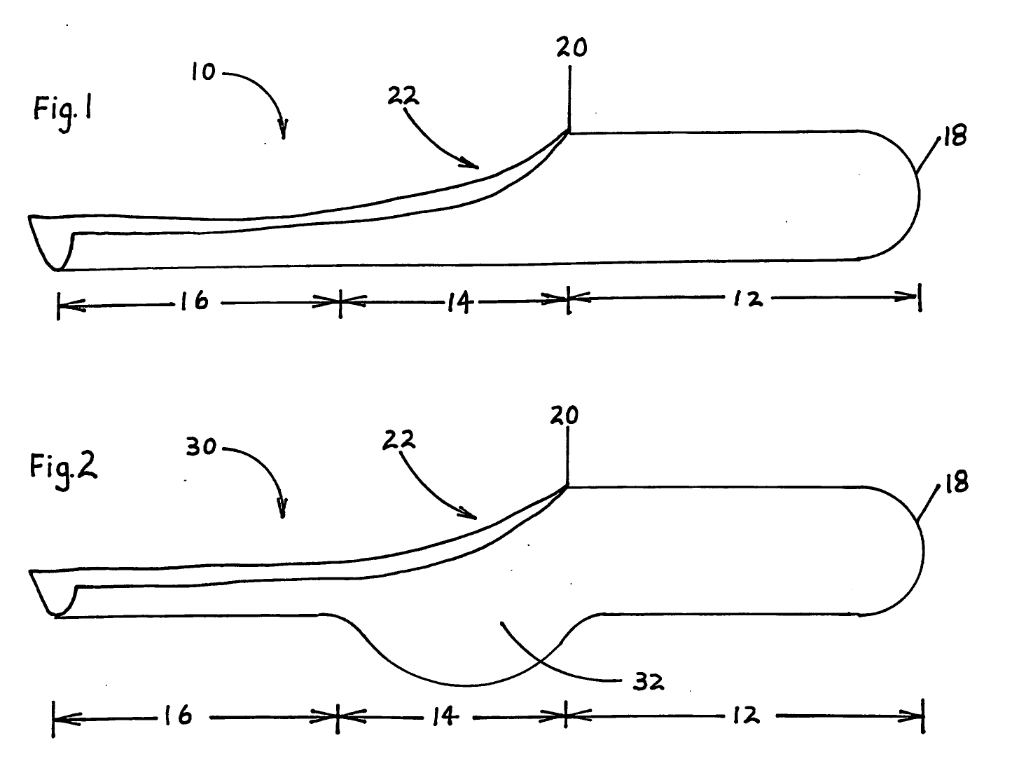

In the alternative embodiment 30 illustrated in FIG. 2, the mid-portion shape in the form of a pouch 32 is to accommodate the scrotum in a looser fashion. (The parts of embodiment 30 corresponding to those of embodiment 10, are indicated by like reference numerals). The pouch 32 receives and supports the scrotum so that it is contained loosely, which allows it to be mor sensitive to external contact and may lead to increased stimulation during sexual activity.

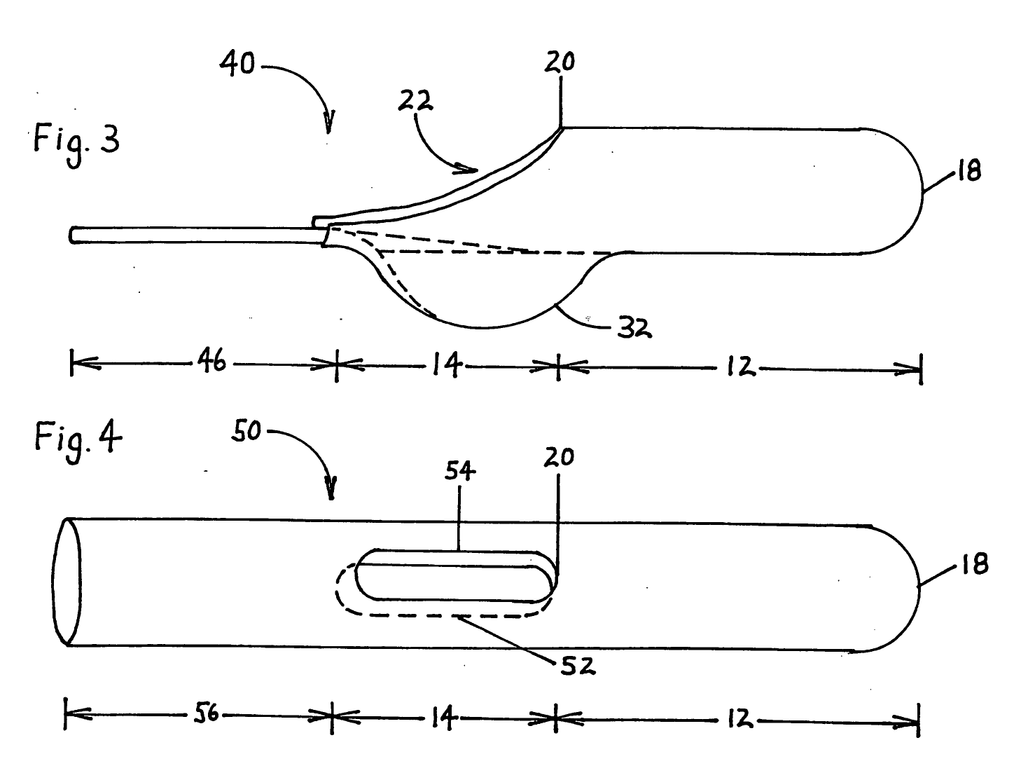

Referring to FIG. 3, a further embodiment 40 of a male sexual aid device is shown. The device 40 also comprises a sheath 12 and a mid-portion 14, but has a tail 46 comprised of a different material, which is attached to or blended with the material of the sheath and mid-portion. The mid-portion can either be in the form of a generally linear extension of the lower side of the sheath as in the FIG. 1 embodiment or in the form of a pouch 32 as in the FIG. 2 embodiment.

The tail 46 may be of a rigid or semi-rigid material capable of being retained between the buttocks of the user. Alternatively, the tail 46 may be of a material that is specially textured to enhance stimulation of nerves in the anal area, providing increased sexual stimulation. For novelty purposes, the tail 46 may be provided with extensions, extending from the rear of the user; for example, an extension simulating the tail of an animal.

As described in the first embodiment, the tail 46 may act as a retention device through the combination of tension or friction. As an alternative, FIG. 6 shows the tail 46 in which a plurality of spaced apertures 26 are present. The device 70 may be provided with a stimulatory anal type plug 100 for insertion in the opening of the anus, further securing the device 40 in place and providing increased sexual stimulation.

In an alternative embodiment of a sexual aid device, generally indicated by reference numeral 50 in FIG. 4, the penis is inserted into the sheath 12 through an opening 54 in the top of the mid-portion 14. When the penis is fully inserted into the sheath 12, the scrotum is placed through the opening 54 in the top of the mid-portion 14 and the opening 52 in the bottom of the mid-portion 14. The tail 56 is then secured between the buttocks as described for the first embodiment, or it can be provided with a stimulatory anal type plug 100 for insertion in the opening of the anus, further securing the device and providing increased sexual stimulation. Placing the scrotum through openings 54 and 52 provides an added method for retaining the device 50 in place and may provide the user with increased comfort from cooler testicles. In this embodiment, increased sensations on the scrotum may be foregone and security against the transmission of STDs through viral shedding of the skin or open lesions will be lessened, but for some men the constriction will provide further sexual gratification.

The device can be included as part of a sexual aid kit. In addition to the device, the kit would include at least one stimulatory anal type plug. The kit could also contain novelty attachments, such as an extension simulating the tail of an animal.

Although certain preferred embodiments of the present invention have been shown and described in detail, it should be understood that various changes and modifications may be made therein without departing from the scope of the appended claims.

What is claimed is:

1. A male sexual aid device comprising:
a sheath of flexible material for loosely covering the penis of a user, said sheath having a closed front end and a rear end, said rear end having an opening therein;
a mid-portion extending rearward from said rear end for covering the scrotum of a user; and
an elongated tail extending rearward from said mid-portion for insertion between the buttocks of said user thereby retaining said device in position on the user.

2. The device according to claim 1, wherein said sheath, said mid-portion and said tail are integrally formed of a flexible material.

3. The device according to claim 1, wherein said sheath and said mid-portion are integrally formed of a flexible material and the tail is formed of a different material which is attached to, or blended with, said mid-portion.

4. The device according to claim 2 or 3, wherein said mid-portion is in the form of a pouch for receiving said scrotum therein.

5. The device according to claim 2 or 3, wherein said flexible material is a plastic, or a latex rubber, polyurethane or other viral blocking material.

6. The devic according to claim 2 or 3, wherein said mid-portion is in the form of a pouch for receiving said scrotum therein and wherein said flexible material is a plastic, or a latex rubber, polyurethane or other viral blocking material.

7. The device according to claim 2 or 3, wherein said flexible material is a plastic, or a latex rubber, polyurethane or other viral blocking material and wherein said device is provided with a lubricant for lubricating the inside of the sheath.

8. The device according to claim 2 or 3, wherein said mid-portion is in the form of a pouch for receiving said scrotum therein, wherein said flexible material is a plastic, or a latex rubber, polyurethane or other viral blocking material and wherein said device is provided with a lubricant for lubricating the inside of the sheath.

9. The device according to claim 2 or 3 wherein said tail is provided with a plurality of spaced apertures.

10. The device according to claim 2 or 3 wherein said mid-portion is in the form of a pouch for receiving said scrotum therein, and wherein said tail is provided with a plurality of spaced apertures.

11. The device according to claim 2 or 3 wherein said tail is provided with a plurality of spaced apertures, wherein said flexible material is a plastic, or a latex rubber, polyurethane or other viral blocking material and wherein said device is provided with a lubricant for lubricating the inside of the sheath;

12. The device according to claim 2 or 3 wherein said mid-portion is in the form of a pouch for receiving said scrotum therein, wherein said tail is provided with a plurality of spaced apertures, wherein said flexible material is a plastic, or a latex rubber, polyurethane or other viral blocking material and wherein said device is provided with a lubricant for lubricating the inside of the sheath;

13. A male sexual aid device comprising:
a sheath of flexible material for covering the penis of a user, the sheath having a closed front end and a rear end, said rear end having an opening therein;
a mid-portion extending rearward from said rear end;
a top opening and a bottom opening, both located in said mid-portion for insertion of said penis through said top opening into said sheath and placement of the scrotum of said user through said top opening and said bottom opening such that said scrotum hangs through said bottom opening;
an elongated tail extending rearward from said mid-portion for insertion between the buttocks of said user thereby retaining said device in position on said user;
wherein said sheath, said mid-portion and said tail are integrally formed of a flexible material; and
wherein said flexible material is a plastic, or a latex rubber, polyurethane or other viral blocking material.

14. The device according to claim 13, wherein said tail is provided with a plurality of spaced apertures.

15. The device according to claim 13 or 14, wherein said device is provided with a lubricant for lubricating the inside of said sheath.

16. A method for installing and retaining a male sexual aid device on a user comprising:
providing a device having a sheath, a mid-portion and an elongated tail extending rearward therefrom;
inserting said user's penis into said sheath; and
inserting said tail between said user's buftocks.

17. A method for installing and retaining a male sexual device on a user comprising:
providing a device having a sheath, a mid-portion and an elongated tail extending rearward therefrom, said tail being provided with a plurality of spaced apertures;
attaching a stimulatory anal type plug to one of said apertures best suited to said user's dimensions;
inserting said user's penis into said sheath; and
inserting said plug in the anus of said user.

18. A sexual aid kit comprising:
a male sexual aid device comprising a sheath, a mid-portion and an elongated tail, said tail being provided with a plurality of spaced apertures; and
a stimulatory type plug for attachment to said device and insertion in the anus of a user of said device, said plug having a portion dimensioned to be releasably inserted into one of said apertures.
